(19) United States
(12) Patent Application Publication
(10) Pub. No.: US 2007/0219415 A1
(43) Pub. Date: Sep. 20, 2007

(54) Interoperative monitoring of intramuscular pressure during retraction

(76) Inventor: Eric Steven Heinz, Memphis, TN (US)

Correspondence Address: KRIEG DEVAULT LLP, ONE INDIANA SQUARE, SUITE 2800, INDIANAPOLIS, IN, 46204-2709, US

(21) Appl. No.: 11/375,627

(22) Filed: Mar. 14, 2006

(51) Int. Cl. A61B 1/32

(52) U.S. Cl. 600/201

(57) ABSTRACT

A method of performing a surgical procedure at an anatomical location in a patient is disclosed. An incision is made in the patient to provide access to an anatomical element. A pressure measurement device may then be inserted into a predetermined area of tissue surrounding the anatomical element. Tissue surrounding the anatomical element may then be retracted with a retractor to expose the anatomical element. A pressure reading from the pressure measurement device may then be obtained that is indicative of an amount of pressure experienced by the predetermined area of tissue as a result of the retraction.

### BACKGROUND

1. Technical Field

The present invention relates generally to monitoring pressure during surgery and more particularly, to a method of monitoring intramuscular pressure during retraction of the muscle tissue surrounding a particular anatomical element of interest.

2. Related Art

Spinal fusion surgery is typically performed on patients to eliminate motion at a vertebral segment in the spine. The procedure can be performed to decrease or eliminate pain that is caused at the vertebral segment located at the point of surgery. Spinal fusion surgery may involve adding bone graft to an area of the spine to set up a biological response that causes the bone graft to grow between two vertebral segments. The growth of the bone graft between the two vertebral segments eliminates motion at that vertebral segment, thereby eliminating the pain caused by that particular vertebral segment.

At every vertebral level in the spine, there is a disc space in the front and paired facet joints in the back. Working together, these respective anatomical structures define a motion segment of the spine and permit multiple degrees of motion. In spinal fusion surgery, typically two or more vertebral bodies are fused together to stop the motion at the vertebral segment or segments that are causing the pain. For example, if a patient is experiencing pain at lumbar segment five, lumbar segment five and lumbar segment six may be fused together in order to eliminate the pain being experienced at lumbar segment five.

Spinal fusion surgery can be performed anteriorly, posteriorly, laterally, or obliquely through an operative approach to the spine. Combinations of these approaches are also possible. In addition to placement of graft and device in one or more spaces between vertebral bodies, plates, rods, staples or other orthopedic implants can be positioned along and secured to two or more vertebrae until fusion is complete. Other spinal procedures involving dynamic spinal stabilization, repair of tissue, replacement of bony and tissue structures, for example, employ one or more operative approaches to the spine.

A suggested cause of adjacent level disc disease after open spinal fusion and other procedures focuses on the weakening of the paraspinal muscles surrounding the spine. The paraspinal muscles are weakened because they are placed in a retracted state during the surgical procedure. As known in the art, retractors are used to retract tissue, including paraspinal muscles, surrounding a portion of the spine that will be operated on during surgery. Retraction causes an increase in the intramuscular pressure, which may lead to injury of nerves and vascular structures of the spine. A pseudo compartment syndrome may be the result, which may cause muscle atrophy, parasthesia, as well as pain.

As such, a need exists for a method of continuously monitoring the amount of pressure experienced by tissue surrounding the spine during spinal surgery so that an excessive amount of pressure is not applied to the tissue during the surgical procedure.

### SUMMARY

A method of monitoring pressure experienced by tissue surrounding a predetermined anatomical element is disclosed. The method may comprise the step of making an incision at a predetermined location surrounding the anatomical element. A pressure measurement device may then be inserted into a selected region or portion of the tissue surrounding the anatomical element. At various locations in the body, the amount of pressure experienced by tissue surrounding the anatomical element being operated on may have dramatic effects on the ultimate outcome or result of the surgery. The pressure measurement device may be used to monitor and record the amount of pressure experienced by these locations in the body during surgical procedures to ensure that too much pressure is not applied.

During the medical procedure, an indication may be communicated to the surgeon to report any abnormal pressure readings from the retraction process. The surgeon may adjust the amount of retraction in response to any abnormal pressure reading. In the alternative, the surgeon may shorten a length of time the muscle tissue is retracted at a particular pressure level in response to any abnormal pressure reading. The pressure reading may also be recorded throughout the procedure in an electronic database file or as a paper printout.

The pressure measurement device may be selected from a group of pressure measurement devices consisting of a needle or catheter and a transducer-tipped pressure monitoring catheter. The output of the pressure measurement device may be connected with a pressure indicator. The pressure indicator may be selected from a group of pressure indicators consisting of a manometer, a digital manometer, a pressure dome, and an electronic display.

Other systems, methods, features and advantages of the invention will be, or will become apparent to one with skill in the art upon examination of the following figures and detailed description. It is intended that all such additional systems, methods, features and advantages be included within this description, be within the scope of the invention, and be protected by the following claims.

### BRIEF DESCRIPTION OF THE DRAWINGS

The components and/or elements in the figures are not necessarily to scale, emphasis instead being placed upon illustrating the principles of the invention. Moreover, in the figures, like reference numerals designate corresponding parts throughout the different views.

FIG. 1 depicts a cross section of a portion of a human spine and surrounding tissue during an open spinal fusion procedure.

FIG. 2 depicts a portion of a human vertebrae of the spine.

FIG. 3 illustrates an output of a pressure measurement device connected with a manometer.

FIG. 4 illustrates an output of a pressure measurement device connected with a pressure dome and an electronic display unit.

FIG. 5 illustrates a transducer-tipped catheter.

FIG. 6 illustrates an output of the transducer-tipped catheter connected with an electronic display unit.

FIG. 7 is a flow chart illustrating representative steps performed during a medical procedure utilizing the present invention.

### DETAILED DESCRIPTION OF THE PRESENTLY PREFERRED EMBODIMENTS

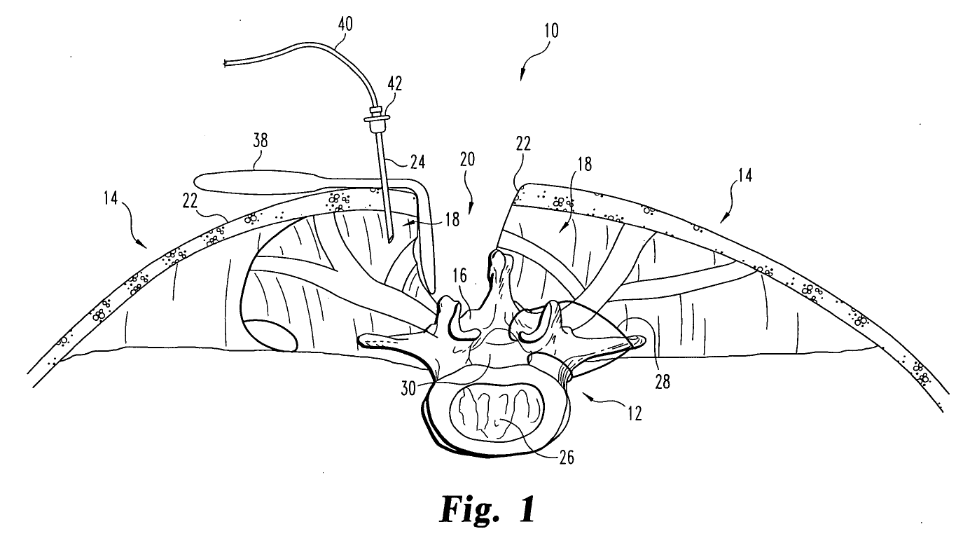

Referring to FIG. 1, a cross section of a portion of a human body 10 is depicted during a surgical procedure, which is illustrated as an open spinal fusion procedure in this embodiment of the present invention. The portion of the human body 10 illustrated comprises a portion of a human spine or vertebral column 12 and a portion of tissue 14 that surrounds the spine 12. The spine 12 is formed, in humans, from twenty-six irregular bones or vertebrae 16 interconnected to one another to form a flexible, curved spine 12. The human spine 12 extends from the skull to the pelvis and includes tissue 14 that surrounds the spine 12.

The spine 12 surrounds and protects a delicate spinal cord and provides attachment points for, amongst other things, paraspinal muscles 18. Paraspinal muscles 18 provide support to the spine 12 and comprise the motor for movement of the spine 12. There are many paraspinal muscles 18 in the human back surrounding and/or attached with the spine 12 at numerous locations. Each one of these muscles 18 controls some part of the total movement between vertebrae 16 and the rest of the skeleton of the human body 10.

During a spinal fusion procedure, an incision 20 may be made in a predetermined amount of integument or skin 22, as well as the tissue 14 surrounding the spine 12, which may include paraspinal muscles 18. The incision 20 provides access to a predetermined area of the spine 12, which is typically located where the bone graft is going to be added to connect respective vertebrae 16 to one another during a spinal fusion procedure. The incision 20 may also be provided for access to spine 12 for attachment of plates, rods or other constructs or for placement of artificial disc devices between vertebrae 16. Although other types of tissue 14 may surround the spine 12, such as skin 22, paraspinal muscles 18 are located at the area of the spine 12 undergoing surgery in this embodiment of the present invention.

While a posterior approach to the spine 12 is shown, anterior approaches are also contemplated. For example, the psoas muscle in the abdominal space can be sensitive to retraction, and monitoring of the pressure applied during retraction can provided the surgeon with information to limit or prevent trauma thereto. Other applications in spinal surgery include lateral and oblique approaches to the spine. In addition, the present invention may have application in other orthopedic surgeries where muscle tissue is retracted.

A pressure measurement device 24 may be placed or inserted into a predetermined location of the paraspinal muscle 18 surrounding the area of interest to monitor pressure applied to this location during the procedure. As previously set forth, the paraspinal muscles 18 provide support to the spine 12 and are the motor for movement of the spine 12. While in a retracted state during surgery, if too much pressure is used to separate the paraspinal muscles 18 surrounding the spine 12 to provide a working surgical area, this may lead to injury of nerves and vascular structures of the spine 12. As set forth in greater detail below, if the amount of pressure applied exceeds a predefined threshold or is held at one particular pressure level for too long, an indicator may be provided to the surgeon so that appropriate action may be taken by the surgeon.

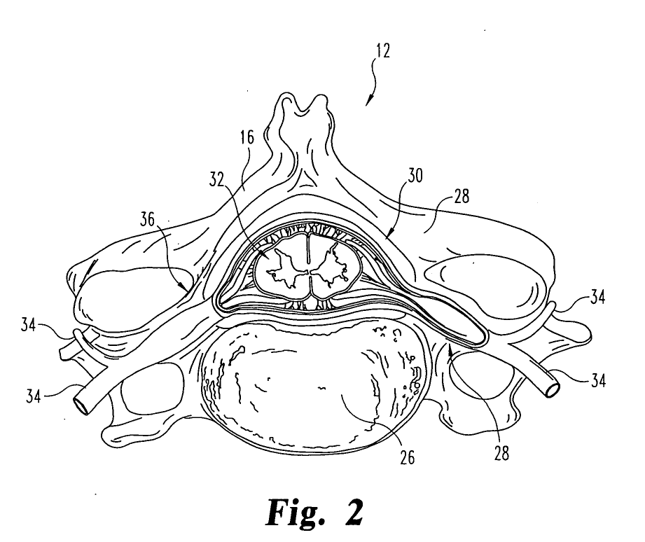

Referring to FIG. 2, generally speaking, the vertebrae 16 of the spine 12 share a somewhat common structural pattern. A vertebrae 16 may include a body 26, anteriorly, and vertebral arches 28, posteriorly. The disc shaped body 26 is the weight-bearing region. The body 26 and the vertebral arches 28 enclose an opening called the vertebral foramen 30. Successive vertical foramen 30 of the articulated vertebrae 16 form the long vertebral canal or spine 12, through which passes a spinal cord 32.

A plurality of spinal nerves 34 may be connected with the spinal cord 32. Through the spinal nerves 34 that attach to it, the spinal cord 12 is involved in the sensory and motor innervation of the entire human body 10 inferior only to the head. Thirty-one pairs of spinal nerves 34 arise from the spinal cord 12 as nerve roots and exit through a intervertebral foramina 36 to travel to the body regions they serve. As known by those skilled in the art, damage to a spinal nerve 34 may affect a patient or a particular region of the body. As such, during spinal surgery it is important not to damage any spinal nerves 34.

Referring back to FIG. 1, once the incision 20 has been made, the pressure measurement device 24 may be inserted into a predetermined area of tissue 14 surrounding the spine 12. In particular, the pressure measurement device 24 may be placed in the paraspinal muscles 18 located at the chosen location of the spine 12. The pressure measurement device 24 is placed in a location such that a pressure reading of the paraspinal muscles 18 may be obtained. This allows the surgeon to monitor the amount of pressure experienced by the paraspinal muscle 18 surrounding the area of surgery, thereby allowing the surgeon to minimize side effects that may occur as a result of an open spinal fusion procedure.

A spinal retractor 38 may be inserted into the incision 22 for retracting the tissue 14 surrounding the spine 12, thereby exposing the spine 12 so that the surgeon can have clear access to the location requiring surgical attention. A pressure reading may be obtained from the pressure measurement device 24 that is indicative of an amount of pressure experienced by the predetermined area of paraspinal muscle 18 as a result of the retraction procedure. The pressure measurement device 24 may include an output 40, which, as set forth below, may be connected with a pressure indication device or indicator.

The pressure measurement device 24 may comprise a needle or catheter. As known in medicine, a catheter is a tube that can be inserted into a body cavity duct or vessel, such as a paraspinal muscle 18. The needle or catheter may include a hollow interior and an opening that allows fluid to pass through the hollow interior. The needle or catheter may need to be bled out to remove any atmospheric fluid contained in the hollow interior or tube. The output 40 may comprise a tube that is connected with a connector 42 of the catheter. As set forth in detail below, the catheter allows the direct measurement of pressure experienced by the paraspinal muscle 18 to be taken during the surgical procedure.

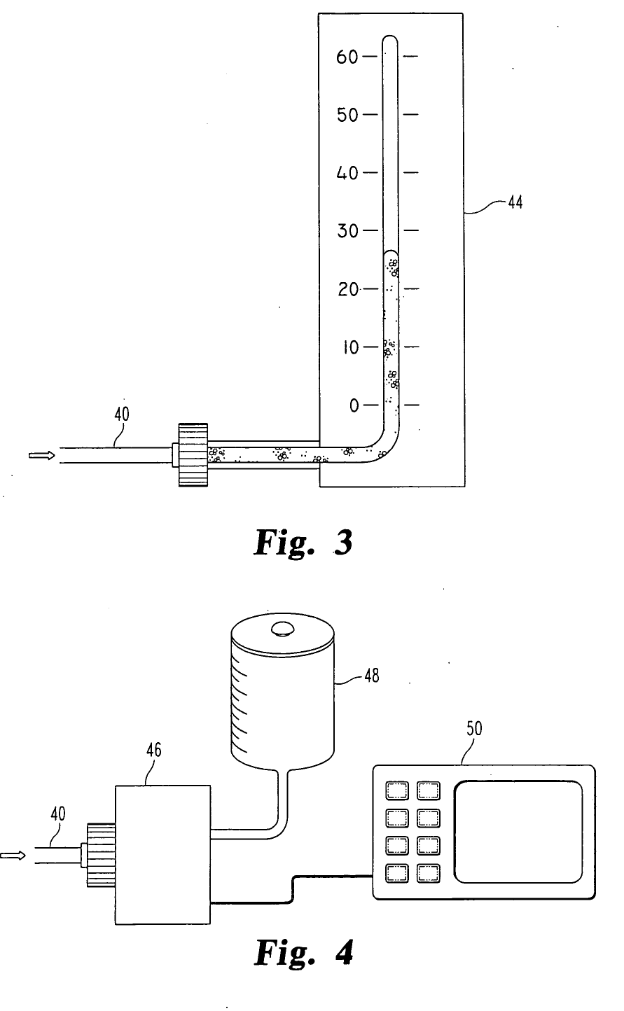

Referring to FIG. 3, the output 40 of the pressure measurement device 24 may be connected with a manometer 44. A manometer 44 is a device commonly employed to measure pressure. Generally speaking, pressure (P) is equal to the ratio of the force (F) applied to a surface to the surface area (A) of the surface. As pressure is applied to the paraspinal muscle 18, by exerting force on the retractor 38, the pressure experienced by the paraspinal muscle 18 may be monitored on the manometer 44. This is because fluid in the output 40 of the pressure measurement device 24, once being inserted into the paraspinal muscle 18, experiences a pressure increase or decrease as a function of the amount of pressure being applied to the paraspinal muscle 18 during retraction.

Referring to FIG. 4, the output 40 of the pressure measurement device 24 may also be connected with a pressure transducer 46. The pressure transducer 46 may be a fluid-to-air pressure transducer that converts fluid pressure to air pressure. A pressure dome 48 or an electronic display unit 50 may be connected with an output of the pressure transducer 46. The pressure dome 48 and the electronic display unit 50 may be operable to convert air pressure into a pressure reading. As such, the pressure being experienced by the paraspinal muscles 18 at the point of interest during surgery may be monitored on the pressure dome 48 or the electronic display unit 50.

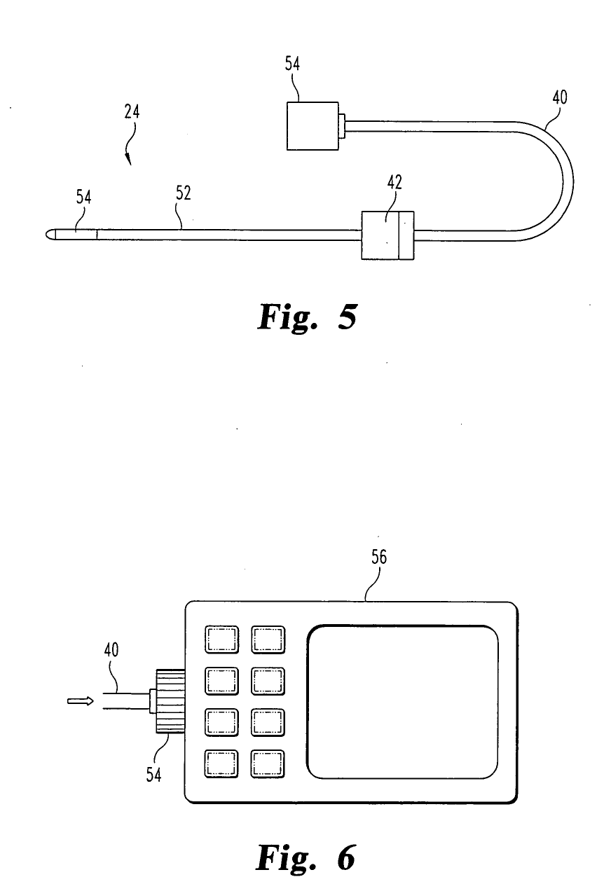

Referring to FIG. 5, the pressure measurement device 24 may comprise a catheter 52 having a pressure transducer 54 disposed at a distal end of the catheter 52. The pressure transducer 54 may be operable to generate an electrical signal indicative of the amount of pressure being experienced by the paraspinal muscle 18 during surgery. The transducer-tipped pressure monitoring catheter 52 may include a connector 42 that connects the catheter 52 to the output 40. The output 40 may be connected with a connector 54 and may comprise electrical wires housed in a tube or sheath in this embodiment. As illustrated in FIG. 6, the connector 54 of the catheter 52 may be connected with an electronic display unit 56 that is operable to display a pressure reading received from the catheter 52. The pressure transducer may be linked to a side port, slit catheter, or simple needle which is placed into the muscle belly.

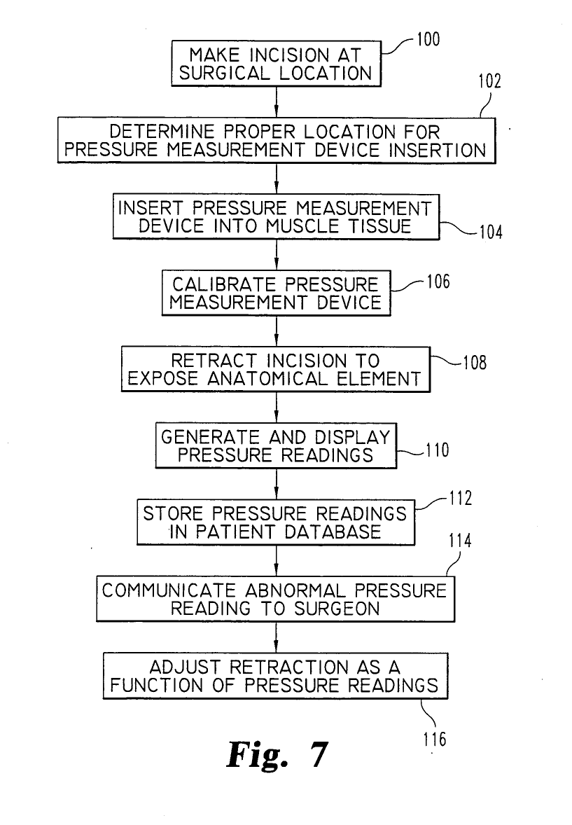

Referring to FIG. 7, a flow chart is set forth that illustrates representative steps that may be performed during open spinal fusion procedures as it relates to the present invention. At step 100, a surgeon may make an incision 20 at a predetermined area in the human body 10 containing an anatomical element of interest (e.g.—spine 12). At step 102, the surgeon may determine a proper location for insertion of a pressure measurement device 24 as a function of muscle tissue surrounding the anatomical element of interest (e.g.—paraspinal muscle 18). Although the preferred embodiment discusses use of the present invention in relation to open spinal fusion procedures, it is envisioned that other types of medical procedures in which pressure needs to be monitored in muscle tissue or other tissue during retraction to prevent damage to surrounding tissue would benefit from the present invention as well.

Once the proper anatomical location is found by the surgeon, the pressure measurement device 24 may be inserted into the tissue of interest. In alternative embodiments, the pressure measurement device 24 may be inserted into the tissue area of interest before the incision 20 is made in the body 10. At step 106, after being inserted, the pressure measurement device 24 may be connected with a pressure indicator (e.g.—manometer 44, pressure transducer 46, pressure dome 48, electronic display unit 50, 56) and may then be calibrated for the medical procedure. The surgeon may then retract the incision 20 and surrounding tissue 14 to expose the anatomical area of interest using a retractor 38, which is illustrated at step 108.

At step 110, a pressure reading is generated as a function of the retraction of tissue performed at step 108. The pressure reading may then be displayed to the surgeon using one of the pressure displays disclosed herein or any other suitable pressure display device. The pressure reading may continuously update throughout the surgical procedure thereby providing the surgeon with real-time pressure indication feedback. At step 112, the pressure readings taken may be stored in an electronic database associated with the patient. This allows the surgeon to verify, at a later date, that pressure levels were maintained at proper and accepted levels throughout the procedure.

If the pressure reading goes above a maximum threshold value (e.g.—30 mm Hg), at step 114, the detection of an abnormal pressure reading may be communicated to the surgeon (e.g.—audible alarm, assistant notification, flashing display or light, and so forth). At step 116, the surgeon may adjust the amount of retraction in order to lower the amount of pressure experienced by the tissue. Communication of an abnormal pressure reading may also occur as a result of the tissue being held at a predetermined pressure level for a given period of time in addition to exceeding a threshold pressure value.

While the present invention has been described with reference to specific exemplary embodiments, it will be evident that various modifications and changes may be made to these embodiments without departing from the broader spirit and scope of the invention as set forth in the claims. Accordingly, the specification and drawings are to be regarded in an illustrative rather than a restrictive sense.

What is claimed is:

1. A method of performing a surgical procedure at an anatomical location in a patient, comprising the steps of:
a) making an incision in said patient to provide access to an anatomical element;
b) inserting a pressure measurement device into a predetermined area of tissue surrounding said anatomical element;
c) retracting tissue surrounding said anatomical element with a retractor to expose said anatomical element; and
d) obtaining a pressure reading from said pressure measurement device indicative of an amount of pressure experienced by said predetermined area of tissue as a result of said retraction.

2. The method of claim 1, further comprising the step of communicating to a surgeon performing said retraction any abnormal pressure readings from step (d).

3. The method of claim 2, further comprising the step of adjusting said retraction of said tissue in response to any abnormal pressure reading.

4. The method of claim 2, further comprising the step of shortening a length of time said tissue is retracted in response to any abnormal pressure reading.

5. The method of claim 1, further comprising the step of recording said pressure reading throughout said surgical procedure.

6. The method of claim 5, wherein said recording is stored in a database associated with said patient.

7. The method of claim 1, wherein said pressure measurement device may be selected from a group of pressure measurement devices consisting of a needle, a catheter, and a pressure transducer catheter.

8. The method of claim 1, further comprising the step of connecting an output of said pressure measurement device to a pressure indicator.

9. The method of claim 8, wherein said pressure indicator may be selected from a group of pressure indicators consisting of a manometer, a digital manometer, a pressure dome, and an electronic display.

10. A method of performing a surgical procedure at an anatomical location in a patient, comprising the steps of:
a) making an incision in said patient to provide access to an anatomical element;
b) inserting a catheter having a hollow interior and an opening into a predetermined area of muscle tissue located in close approximation to said anatomical element, wherein said catheter is connected with a first end of a tube;
c) connecting a second end of said tube with a pressure detection device;
d) retracting muscle tissue surrounding said anatomical element with a retractor to expose said anatomical element; and
e) obtaining a pressure reading from said pressure detection device indicative of an amount of pressure experienced by said predetermined area of muscle tissue as a result of said retraction.

11. The method of claim 10, further comprising the step of bleeding atmospheric fluid from said tube prior to step (c).

12. The method of claim 10, further comprising the step of communicating to a surgeon performing said retraction any abnormal pressure readings from step (e).

13. The method of claim 12, further comprising the step of adjusting said retraction of said muscle tissue in response to any abnormal pressure reading.

14. The method of claim 12, further comprising the step of shortening a length of time said muscle tissue is retracted in response to any abnormal pressure reading.

15. The method of claim 10, further comprising the step of recording said pressure reading in an electronic database continuously throughout said surgical procedure.

16. The method of claim 10, further comprising the step of connecting an output of said pressure measurement device to a pressure indicator.

17. The method of claim 10, further comprising the step of displaying said pressure reading to a surgeon.

18. A method of performing a surgical procedure at an anatomical location in a patient, comprising the steps of:
a) making an incision in said patient to provide access to an anatomical element;
b) inserting a catheter having a pressure transducer disposed on a shaft portion of said needle into a predetermined area of muscle tissue surrounding said anatomical element;
c) retracting muscle tissue surrounding said anatomical element with a retractor to expose said anatomical element; and
d) obtaining a pressure reading from said pressure transducer indicative of an amount of pressure experienced by said predetermined area of muscle tissue as a result of said retraction.

19. The method of claim 18, further comprising the step of communicating to a surgeon performing said retraction any abnormal pressure readings from step (d).

20. The method of claim 19, further comprising the step of adjusting said retraction of said muscle tissue in response to any abnormal pressure reading.

21. The method of claim 19, further comprising the step of shortening a length of time said muscle tissue is retracted in response to any abnormal pressure reading.

22. The method of claim 18, further comprising the step of recording said pressure reading in an electronic database throughout said surgical procedure.

23. The method of claim 18, further comprising the step of connecting said catheter to a pressure indicator.

24. The method of claim 23, wherein said pressure indicator comprises an electronic display.
